(19) United States
(12) Patent Application Publication
(10) Pub. No.: US 2022/0151510 A1
(43) Pub. Date: May 19, 2022

(54) USER DETECTION AND IDENTIFICATION IN A BATHROOM SETTING

(71) Applicant: TOI LABS, INC., San Francisco, CA (US)

(72) Inventors: Vikram Kashyap, San Francisco, CA (US); Paul Cristman, San Francisco, CA (US)

(21) Appl. No.: 17/432,955

(22) PCT Filed: Feb. 22, 2020
(86) PCT No.: PCT/US2020/019383

§ 371 Date: Aug. 23, 2021

Related U.S. Application Data
(60) Provisional application No. 62/809,522, filed on Feb. 22, 2019.
(60) Provisional application No. 62/900,309, filed on Sep. 13, 2019.
(60) Provisional application No. 62/959,139, filed on Jan. 9, 2020.

(51) Int. Cl. A61B 5/117; G01N 33/493; G01N 31/22; A61B 10/00; G06V 10/74; H04N 7/18

(52) U.S. Cl. CPC A61B 5/117; G01N 33/493; G01N 31/22; G01S 17/894; A61B 10/007; G06V 10/74; H04N 7/188; A61B 10/0038

(57) ABSTRACT

Provided is a system for detecting a user of a bathroom. The system comprises at least one sensor coupled to a bathroom use analysis device, where the sensor generates data that can be used to detect and/or identify the user. Also provided is a method of detecting a user of a bathroom. The method comprises analyzing data generated by the sensor in the above system to detect and/or identify the user.

### CROSS-REFERENCE TO RELATED APPLICATIONS

This application claims the benefit of U.S. Provisional Application No. 62/809522, filed Feb. 22, 2019, U.S. Provisional Application No. 62/900309, filed Sep. 13, 2019, and U.S. Provisional Application No. 62/959139, filed Jan. 9, 2020. All three applications are incorporated by reference herein in their entirety.

### BACKGROUND OF THE INVENTION

### (1) Field of the Invention

The present application generally relates to methods of detecting and identifying individuals. More specifically, methods and systems for detecting and identifying a user of a bathroom are provided.

### (2) Description of the Related Art

Disclosed in PCT Patent Publication WO 2018/187790 are biometric monitoring devices, methods and systems related to biomonitoring in a bathroom setting. As disclosed therein, it is useful or necessary to detect or identify a user when the devices and systems are used. Provided herein are systems and methods for detecting or identifying a user of a bathroom device.

### BRIEF SUMMARY OF THE INVENTION

Provided is a system for detecting a user of a bathroom. The system comprises at least one sensor coupled to a bathroom use analysis device, where the sensor generates data that can be used to detect and/or identify the user.

Also provided is a method of detecting a user of a bathroom. The method comprises analyzing data generated by the sensor in the above system to detect and/or identify the user.

### BRIEF DESCRIPTION OF THE SEVERAL VIEWS OF THE DRAWINGS

FIG. 1 is a perspective view of a toilet with an excreta analysis device having a user detection component.

FIG. 2 is an exploded view of a user detection component.

FIG. 3 is a perspective view of an excreta analysis device having a user detection component.

FIG. 4 is two perspective views of a toilet with the seat up and with the seat down, the toilet having an excreta analysis device with a user detection component.

FIG. 5 is a flow chart of a user identification system that is coupled to an excreta analysis device.

FIG. 6 is a perspective view of a toilet coupled to various components that are part of a user identification system.

FIG. 7 is a flow chart of steps used by a user identification system to identify a user.

FIG. 8A, 8B, 8C, 8D, 8E, and 8F are views of toilet seats showing sensor configurations.

FIG. 9A are exploded views of a user detection component in a toilet seat lid.

FIG. 9B is a perspective view of a toilet seat lid with a two component user detection system.

FIG. 9C is a cross section view of a two part lid with a user detection component inside.

FIG. 9D is a perspective view of a top lid single component user detection component.

FIG. 10A is a perspective view of an upward facing user detection component that allows for movement in two degrees of freedom.

FIG. 10B is a perspective view of single fixed upward facing user detection component.

FIG. 11 are three perspective views of location for a fingerprint reader or other sensors/user input.

### DETAILED DESCRIPTION OF THE INVENTION

In PCT Patent Publication WO 2018/187790, devices, methods and systems are provided for analyzing a bathroom user's excreta and for performing other tasks in a bathroom, such as weighing a user, dispensing medications to a user, and taking the body temperature of a user. Detection and/or identification of the user of those devices is needed to associate a user with the information captured about the user for purposes such as medication adherence, medication dosage/prescription, compliance (e.g., court ordered drug testing), billing, and obtaining baseline and abnormal results for that user. The present invention addresses that need.

Provided herein is a system for detecting a user of a bathroom. The system comprises at least one sensor coupled to a bathroom use analysis device. In these embodiments, the sensor generates data that can be used to detect and/or identify the user.

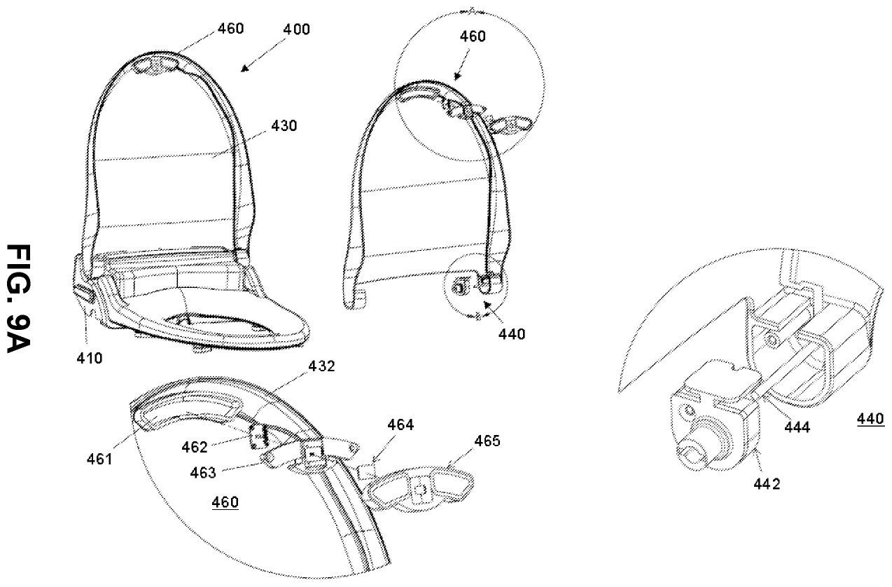

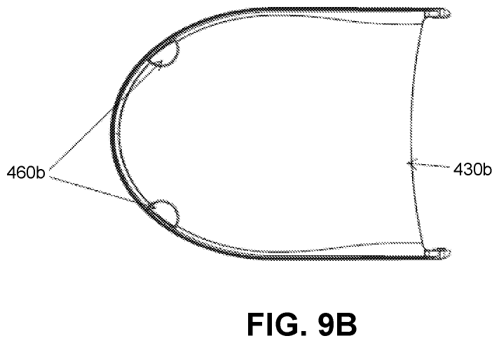

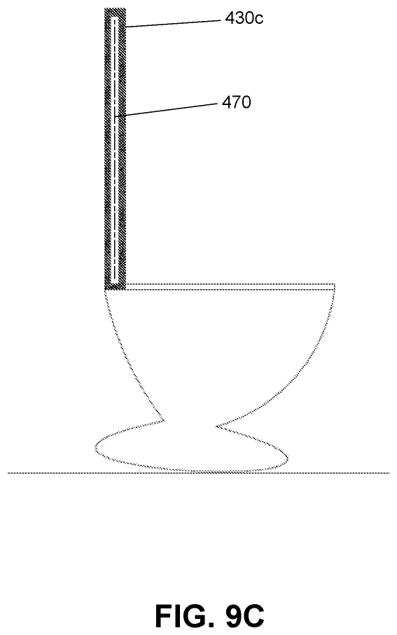

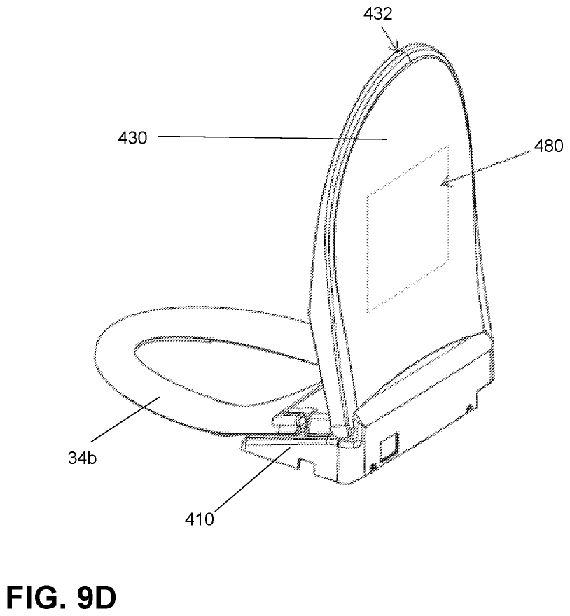

As used herein, a bathroom use analysis device (hereinafter “BUAD”) is a device that measures a parameter of the use of a bathroom appliance such as a sink, a mirror, a tub, a bidet, a shower, a medicine cabinet, or a toilet. For example, the BUAD could capture a facial image from a mirror (see, e.g., p. 10 and FIG. 9A-D of WO 2018/187790); keep track of and/or dispense medicine from a medicine cabinet (see, e.g., p. 10 and FIG. 9A-D of WO 2018/187790), or measure and analyze characteristics of excreta (“excreta analysis device”) in a toilet, e.g., as in multiple embodiments described in WO 2018/187790.

In some embodiments, the system detects the presence of a user but does not identify the user. Those embodiments can be used where the measurements made by the BUAD at that time point are not compared with measurements from other time points.

In other embodiments, the system detects and identifies the user, can distinguish between users, and creates a user profile for each user. These systems allow for evaluation of the user's use of the BUAD over time, and provide diagnostic information when the BUAD obtains an abnormal reading.

The sensor in these systems can be any sensor, now known or later developed, that determines the presence of a user, or measures a characteristic that varies between individuals. Nonlimiting examples include explicit identifiers, image sensors, time of flight cameras, load cells, capacitive sensors, microphones, image sensors, ultrasonic sensors, passive infrared sensors, thermopiles, temperature sensors, motion sensors, photoelectric sensors, structured light systems, fingerprint scanners, retinal scanners, iris analyzers, smartphones, wearable identifiers, scales integrated with a bathroom mat, height sensors, skin color sensors, bioelectrical impedance circuits, electrocardiograms, or thermometers.

The system can comprise multiple sensors, or any combination of sensors, either housed together, or separately connected into the system.

In various embodiments, the system can store a set of identifiers in association with a user. Nonlimiting examples of identifiers that can be utilized to identify a user are explicit identifiers, voice identifiers, image identifiers, structured-light 3D scanning identifiers (e.g., measuring the three-dimensional shape of a face using projected light patterns and a camera system), fingerprint identifiers, retinal identifiers, and smartphone/wearable identifiers further described below.

### Explicit Identifiers

In some embodiments, the system can store a set of explicit identifiers in association with a user profile. An explicit identifier is an identifying input received directly at the BUAD or via the native application executing on a user device. For example, the system can assign a particular button or input on the touchscreen of the BUAD to a particular user and can store the assignment in association with the user profile corresponding to the user. In one implementation, the BUAD can display, via a touchscreen, an input area corresponding to each user profile associated with the bathroom use analysis device. Alternatively, the BUAD can include a set of physical buttons and assign each physical button with a user profile. Therefore, prior to using an appliance in the bathroom, a user may identify herself to the BUAD by interacting with the BUAD or the native application executing on her user device.

### Voice Identifiers

In other embodiments, the system can store a set of voice identifiers in association with a user profile. A voice identifier is an audio clip of the user's voice speaking a particular word or phrase. In some of these embodiments, the system can, during an onboarding process: prompt the user to pronounce her name or another identifying phrase; and record several audio clips of the user pronouncing her name. The system can then, upon detecting a presence of an unknown user, prompt the user to state her name. The system can then record the response to the prompt for voice identification and compare the response to the stored set of voice identifiers associated with the user profile. The system can then utilize voice identification and/or authentication technology to match the response to the set of voice identifiers associated with the user profile.

In various embodiments, the system can prompt the user to repeatedly pronounce an identifying phrase in order to increase the number of voice identifiers associated with the user's profile and thereby increase the likelihood of positively identifying the user.

### Image Identifiers

In additional embodiments, the system can store a set of image identifiers in association with a user profile. An image identifier is a picture of the user such that the system can utilize for recognition purposes.

As used herein, a system that utilizes an image identifier, e.g., using a camera to identify a user, is not narrowly limited to facial detection, but includes any kind of images that can be used to identify a person or distinguish known users from guests, for example images of the body, the back of the user's head, relative shoulder/neck length, etc.

In particular embodiments of these systems, the sensor comprises an image sensor, a time of flight camera, a load cell, a temperature sensor, or any combination thereof. In some of these embodiments, the sensor is an image sensor, e.g., a time-of-flight camera.

As an example, the system can, during an onboarding process, perform any or all of the following tasks: prompt the user to look into a camera integrated into the BUAD (or a camera on the user's smartphone); record multiple images of the user's face; record images of each user prior to a BUAD use and execute face recognition techniques to compare images of the current user to visual identifiers stored in association with the user profile in order to identify the current user of the BUAD. In specific embodiments, the system can also import a preexisting image or set of images from a user device such as the user's smartphone.

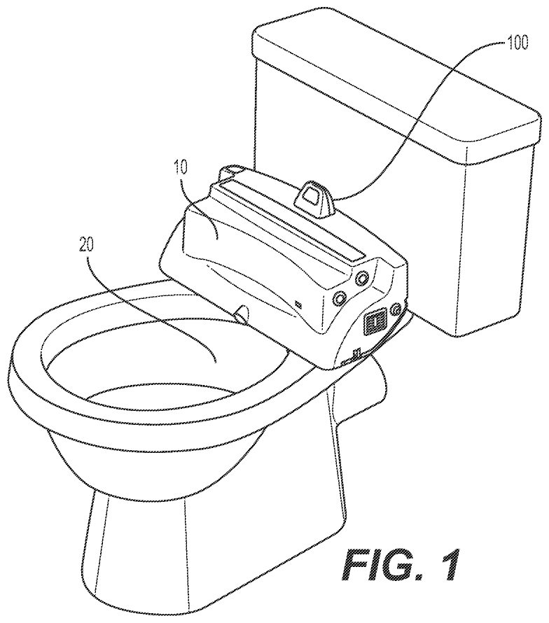

The image sensor in these embodiments can be installed anywhere that can sense a desired image of the user. Nonlimiting examples include a wall-mounted mirror; portable mirror; a toilet paper roll; a sink; a mat in front of a toilet or sink, separately mounted on a wall, above a seat or a seat cover on the toilet; installed or integrated into a seat or a seat cover on the toilet; or integrated or installed onto the seat cover on the toilet, where the image sensor is capable of imaging the user only when the seat cover is raised. See, also, FIGS. 1, 3, and 4; and various embodiments in WO 2018/187790.

In one implementation, the system can prompt the user to vary the angle of her face relative to the camera in order to record a variety of facial images in order to improve the likelihood of identifying the user prior to BUAD use.

In another implementation, the system can prompt the user to approach or position her body to vary the angle and position relative to the camera in order to record a variety of images in order to improve the likelihood of identifying the user prior to BUAD use. In this implementation the system executes gate or posture analysis prior to BUAD use.

In another implementation, the system can prompt the user to wash her hands in the sink in order to record a variety of hand images in order to improve the likelihood of identifying the user prior to BUAD use.

In another implementation, the system can include a set of lighting instruments that the system can activate responsive to detecting the presence of a current user of the excreta analysis device. The system can then record images of the current user with an improved likelihood of identifying the user due to consistent lighting conditions.

Thus, the system can perform any or all of the following: record a first image of a first user in association with the user profile of a first user; during a subsequent BUAD use, record a second image of a current user; and match the second image to the first image to identify the current user as the first user.

In further embodiments, the system can store a set of structured-light/3D scanning identifiers in association with a user profile. A structured-light/3D scanning identifier is a 3D representation of the shape of a user's face or body suitable for identification purposes. For example, the system can, during an onboarding process: prompt the user to look into a camera, structured light system, or 3D scanner; and record a 3D scan of the user's face. The system can then perform a 3D dimensional scan of each user's face prior to an excretion event and execute face recognition techniques to compare a 3D scan of the current users to 3D scans stored in association with the user profile in order to identify the current user of the BUAD.

In another implementation a structured-light/3D scanning identifier is a 3D representation of the ears and back of the user's head suitable for identification purposes.

### Fingerprint Identifier

In some embodiments, the system can store a fingerprint identifier in association with a user profile. A fingerprint identifier is a representation of specific identifiable features (i.e. minutiae) of a user's fingerprint. In these embodiments, an example of an onboarding process is: prompt the user to scan her fingerprint (in several different orientations) at a fingerprint scanner located at the BUAD (e.g., at the flush handle or button of a toilet); and record the user's fingerprint each time the user repositions her finger. The system can then, upon detecting the presence of a current user, prompt the current user to scan her finger at the BUAD in order to identify the user. Alternatively, in implementations of the BUAD including a fingerprint scanner on the flush handle or button of a toilet, the system can record an excretion event and identify the user responsible for the excretion event, upon scanning the user's fingerprint as she flushes the toilet.

### Iris/Retina Identifiers

In additional embodiments, the system can store an iris or retinal identifier in association with the user profile. An iris/retinal identifier is an image or other representation of the user's retina or iris. An example of an onboarding process for these embodiments, is: prompt the user to place her eye in position for a retinal scan located at a retinal scanner proximal to the BUAD; and record an infrared image of the user's retina. Additionally or alternatively, the system can: prompt the user to look into a camera integrated into the BUAD; record high-resolution visual light images of a user's face; and extract images of the user's iris. The system can then, upon detecting the presence of a user, prompt the current user to scan her retina at the retinal scanner or look into a camera integrated with the BUAD in order to record an image of the user's iris.

### Smartphone/Wearable Identifiers

In some embodiments, the system can store a smartphone/wearable identifier in association with a user profile. A smartphone/wearable identifier is a universally-unique identifier (hereinafter “UUID”) for a wireless communication protocol ID associated with a device owned by the user. For example the system can, during an onboarding process prompt the user to synchronize her device(s) with the excreta analysis device and record an ID of the device for the wireless protocol. The user's UUID may be added to the system remotely as part of a group of users. The system can then detect proximity of the device to the excreta analysis device and therefore relate recorded excretion events with a particular user based on the smartphone/wearable identifier. More specifically, the system can broadcast a wireless beacon signal and, upon reception of the wireless beacon, the user device can respond with a UUID. The system can then identify the current user by matching the received UUID with an existing smartphone/wearable identifier stored in association with a user profile.

In various embodiments, for example implementations of the system for use in care facilities (such as hospitals or long-term care facilities), the system can include wearable devices. The system can then store a wearable identifier in association with a user profile for each patient and, upon detecting proximity of the wearable device, associate a BUAD use with the patient associated with the wearable device.

The sensors described above allow the system to record user characteristics measured by the specific sensor(s) employed, which are further described below.

### Total Weight

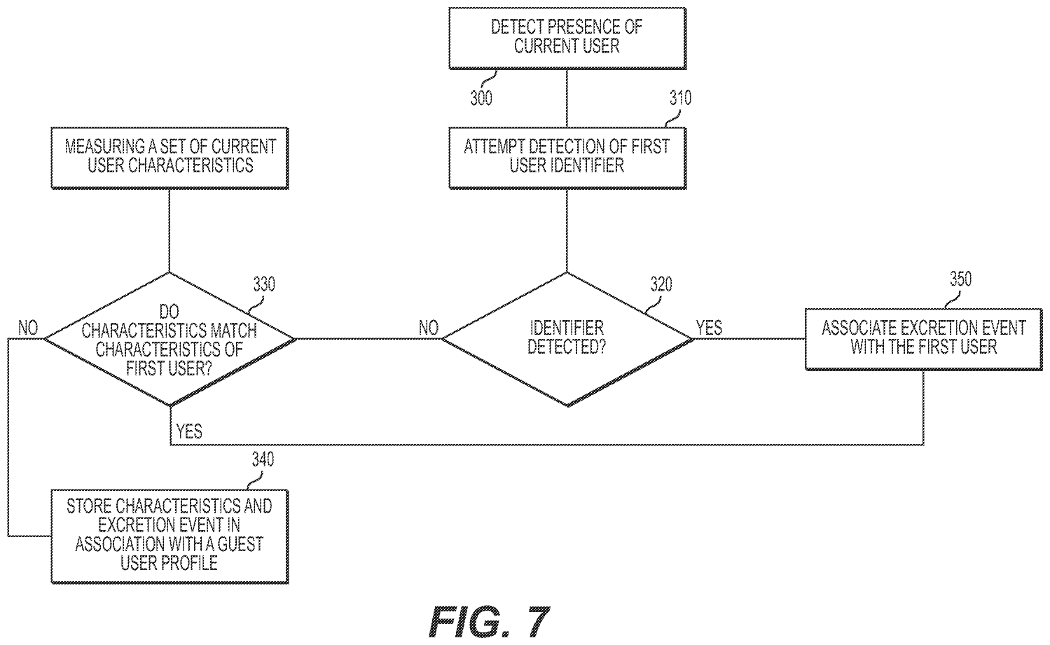

In some embodiments, the system can measure and record the total weight of the user and store the total weight of the user in association with the user profile. In one implementation, the system includes a scale integrated with a bathroom mat, e.g., as described in WO 2018/187790 at p. 9 and FIG. 7, which can include a set of load cells capable of measuring the total weight of the user. Therefore, when a current user is preparing to use the BUAD, the system can measure the weight of the user as the user steps onto the bathroom mat. The system can then compare the weight of the current user to a set of weights stored in association with the user profile in order to increase the likelihood of identifying the user. Thus, the system can: record a first weight in association with a user profile of a first user; during or prior to a subsequent BUAD use, record a second weight of a current user; and match the second weight to the first weight to identify the current user as the first user.

### Seat Load Cell Distribution

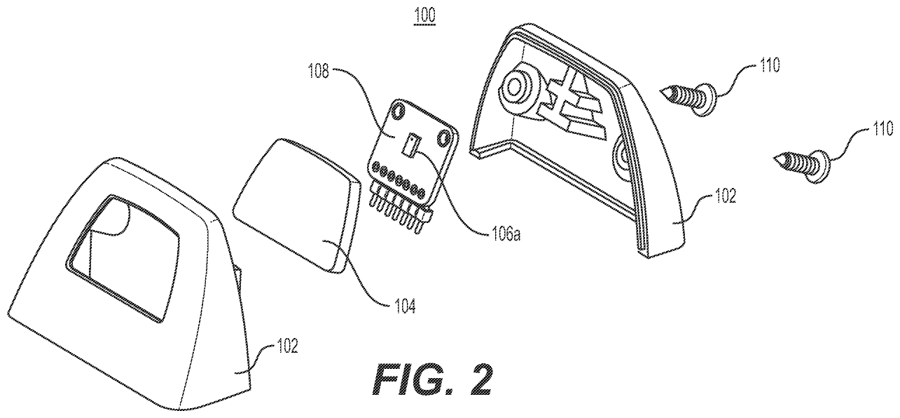

In various embodiments, the system can measure and record a load distribution of the user on a seat in the bathroom, and store the load cell distribution in association with the user profile. In one implementation, the excreta analysis device includes a set of load cells integrated within the toilet seat, e.g., as described in WO 2018/187790 at p. 4 and FIG. 2D. In those embodiments, when a user sits on the excreta analysis device during an excretion event, the system can measure the distribution of force across this set of load cells. Particular users may introduce similar load distributions each time they sit on or stand up from the excreta analysis device, even as their overall weight changes. The load cell signals may be used to look for unique patterns to identify an individual based on changes during an event that occur due to the use of toilet paper. Thus, the exemplified system can perform any or all of the following: record a first load cell distribution in association with a user profile of a first user; during a subsequent BUAD use, record a second load cell distribution of a current user; and match the second load cell distribution to the first load cell distribution to identify the current user of the BUAD as the first user.

### Height

In additional embodiments, the system can measure and record a height of the user and store the height of the user in association with the user profile. In one implementation, the system includes a height sensor (e.g., a visual light, or infrared camera) configured to detect the height of the user as she sits or stands proximal to the BUAD. Thus, the exemplified system can perform any or all of the following: record a first height of the user in association with a user profile of a first user; during or prior to a subsequent BUAD use, record a second height of a current user; and match the second height with the first height to identify the current user as the first user.

### Skin Color

In certain embodiments, the system can record a skin color of the user in association with the user profile. In one implementation, the system can include a skin color sensor (e.g., a low-resolution) visual light camera and LED) configured to detect the skin color of the user upon detecting a user's skin contacting the surface of the BUAD (e.g., on the surface of the toilet seat of an excreta analysis device). Thus, in this example, the system can perform any or all of the following: record a first skin color in association with the user profile; during the use, record a second skin color of a current user of the BUAD; and match the first skin color to the second skin color to identify the current user as the first user.

### Bioelectrical Impedance

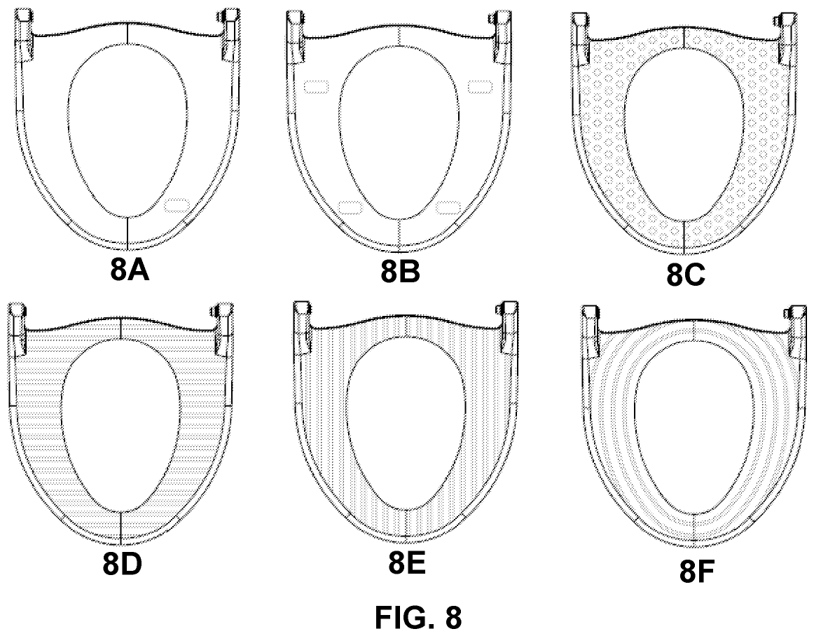

In other embodiments, the system can record a bioelectrical impedance of the user in association with the user profile. The electrodes for bioelectrical impedance can be placed in any useful pattern on the seat or lid. FIG. 8A, 8B, 8C, 8D, 8E, 8F show exemplary patterns. The patterns shown therein can be either on the top or bottom of the seat.

In one implementation, the system can include a bioelectrical impedance circuit (e.g., integrated with the toilet seat of an excreta analysis device) configured to measure the bioelectrical impedance of the user when the user is using the BUAD. The bioelectrical impedance electrodes could be configured in a variety of patterns and use multiple electrodes to improve the measurement. Repeat measurements could be taken over the use of the system to further distinguish the user. Thus, the system can perform any or all of the following: record a first bioelectrical impedance in association with the user profile of a first user; during a subsequent BUAD use, record a second bioelectrical impedance of a current user; and match the second bioelectrical impedance to the first bioelectrical impedance to identify the current user as the first user.

### Heartrate/Electrocardiogram

In additional embodiments, the system can record the heartrate, heartrate variability, or any other detectable characteristic of a user's heartbeat via an electrocardiogram (e.g., utilizing electrodes installed on the BUAD, such as a toilet seat of an excreta analysis device). The heartrate/electrocardiogram electrodes could be configured in a variety of patterns and use multiple electrodes to improve the measurement. Repeat measurements could be taken over the use of the system to further distinguish the user. Thus, the system can perform any or all of the following: record a first heartrate in association with the user profile of a first user; during a subsequent BUAD use, record a second heartrate of a current user; and match the second heartrate to the first heartrate to identify the current user as the first user. In one implementation, the system can record a first electrocardiographic pattern (e.g., comprising average durations of the P wave, PR segment, QRS complex, ST segment, T wave and U wave of the user or the average ration of the PR interval to the QT interval) in association with a first user; during a subsequent BUAD use record a second electrocardiographic pattern; and match the second electrocardiographic pattern to the first electrocardiographic pattern.

### Pulse Oximetry

In additional embodiments, the system can record the heartrate, heartrate variability, or any other detectable characteristic of a user's heartbeat via a pulse oximeter. A number of different optical techniques could be used, for example exciting the skin with two or more wavelengths of light and using a detector to analyze the received signal. Similarly, using a broadband light source and selective filters on the detector could be used to create a pulse oximetry system in the system. Combining optical and acoustic methods known as photoacoustic or optoacoustic imaging techniques could be used to save on cost, power, and/or processing needs. By taking repeated measurements and or multiple measurements during an event could be used to identify different users of the system. The system could be included in one or multiple sensor configurations as shown in FIGS. 8A, 8B, 8C, 8D, 8E, and 8F. Thus, the system can perform any or all of the following: record a first blood oxygenation of a first user in association with the user profile of a first user; during a subsequent BUAD use, record a second blood oxygenation of a current user; and match the second blood oxygenation to the first blood oxygenation to identify the current user as the first user.

### Acoustic Sensors

In additional embodiments, the system can include acoustic, sonic, or ultrasonic, sensors which could be used to identify a person. In one embodiment, the system could include a 1, 1.5, or 2 dimensional ultrasound imaging system to image a user's thigh generating a 2 or 3 dimensional image/volume for identification. Users' ultrasound images could be uniquely identified using a variety of methods such as but not limited to, tissue composition analysis (fat vs muscle vs bone), doppler or flow based analysis, machine learning, or neural networks. Thus, the system can perform any or all of the following: record a first ultrasound image/volume of a first user in association with the user profile of a first user; during a subsequent BUAD use, record a second ultrasound image/volume of a current user; and match the second ultrasound image/volume to the first ultrasound image/volume to identify the current user as the first user.

In additional embodiments, the system can include a single ultrasound transducer that could be used for activation or identification. In one implementation, the system can include a single ultrasound sensor configured to measure the profile and/or thickness of the leg of the user upon detecting a user's skin contacting the surface of the BUAD (e.g., on the surface of the toilet seat of an excreta analysis device). The profile can be compared to the stored users for identification. The change in electrical response of the ultrasound transducer due to contact with the human body can be used to activate the unit. In another implementation, a skin profile could be recorded instead of the entire leg by using a higher frequency ultrasound transducer. In another embodiment, the system could include an acoustic sensor in the audible frequency range to record audio of respiration of the user. From the recording a number of indirect identification information can be recorded e.g., respiration rate, intensity/volume, and/or tone.

### Temperature

In various embodiments, the system can record the temperature via a temperature sensor at the BUAD, e.g., a toilet seat of an excreta analysis device or via an infrared temperature sensor) of a user. Thus, the system can perform any or all of the following: record a first temperature of a first user in association with the user profile of a first user; during a subsequent BUAD use, record a second temperature of a current user; and match the second temperature to the first temperature to identify the current user as the first user.

### Capacitive Sensors

In various embodiments, the system can measure a change in a capacitive sensor as a method of activation and/or identification. In one implementation using a capacitive sensor that covers the entire seat area, the change in the electrical signal from the capacitor is proportional to the areas of the body in contact with the seat. Thus the sensor can be used to distinguish users with different contact areas on the seat, e.g., children from adults. In another implementation, the capacitive sensor can be designed to be sensitive to changes in body composition and/or weight. Thus, the system can perform any or all of the following: record a first capacitance change in association with the user profile of a first user; during a subsequent BUAD use, record a second capacitance change of a current user; and match the second capacitance change to the first capacitance change to identify the current user as the first user. In yet another implementation, the capacitive sensor can register at a certain threshold the presence of the user and activate the BUAD.

### Body Composition

In various embodiments, the system can approximate the body composition via a body composition sensor at the BUAD, e.g., a toilet seat of an excreta analysis device or via a scale or connected floor sensor) of a user. Thus, the system can perform any or all of the following: record a first body composition approximation of a first user in association with the user profile of a first user; during a subsequent BUAD use, record a second body composition approximation of a current user; and match the second body composition approximation to the first body composition approximation to identify the current user as the first user.

Examples of a user detection and/or identification system associated with an excreta analysis device is provided in FIGS. 1-4.

FIG. 1 illustrates an embodiment of an excreta analysis device 10 with an example of a user detection component 100 installed on an exemplary toilet bowl 20.

Details of an exemplified user detection component are illustrated in FIG. 2. Housing 102 contains a lens cover 104 upon which coatings may be present that harden the material, provide anti-reflective properties that allow infrared light to pass through, are hydrophilic, are hydrophobic and/or have anti-smudge properties. An indirect time-of-flight camera module 108 with the sensing element 106a is shown, but any of the other sensors described above may be used. In this embodiment, the housing 102 is held together by screws 110.

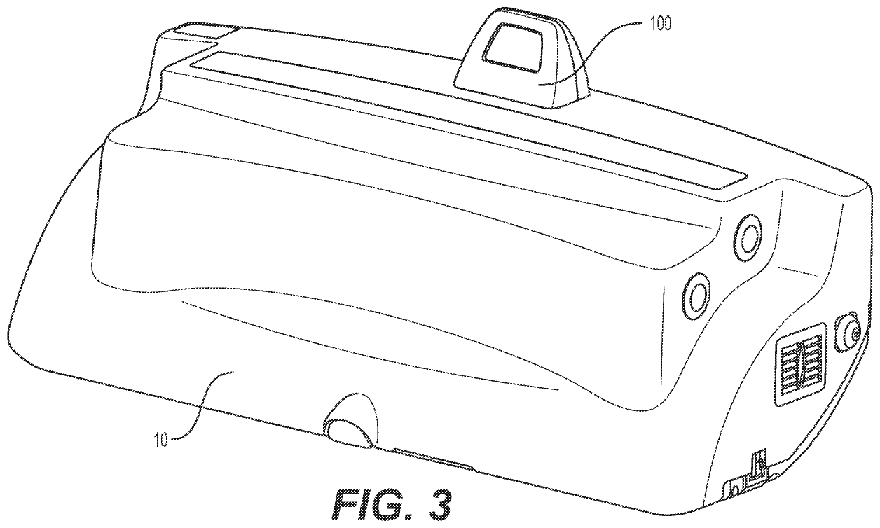

FIG. 3 shows the placement of the illustrated embodiment of the user detection component 100 on an exemplary biomonitoring device 10 that is illustrated in FIG. 2 of WO 2018/187790. The position of the user detection component 100 in the exemplified embodiment allows the detection of user presence while a separate seat, which may be adjustable by height, along with support arms, often known as a commode chair, is used.

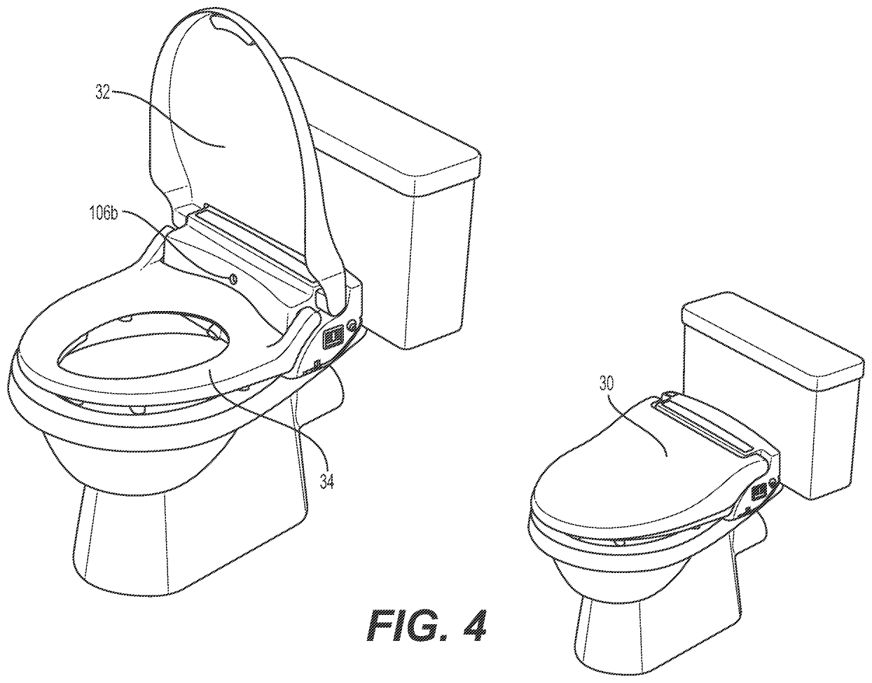

FIG. 4 shows an alternative placement position of the sensor 106b of the user detection component that can be used in conjunction with a raised toilet seat 32 and/or support arms to help a user sit down and get up from the toilet. In the device disclosed in FIG. 2 of WO 2018/187790, seat 34 can be used when a commode chair or other apparatus for helping the user sit down and get up from the toilet is not required. When a user is standing and urinating, the seat cover 32 is up and the position of a sensor 106b that resolves distance is located just above seat level such that when the toilet seat is up, the range of the sensor is not affected by the toilet cover. A sensor that resolves distance is able to detect when toilet cover 30 is in the down position.

### Sensor Positions

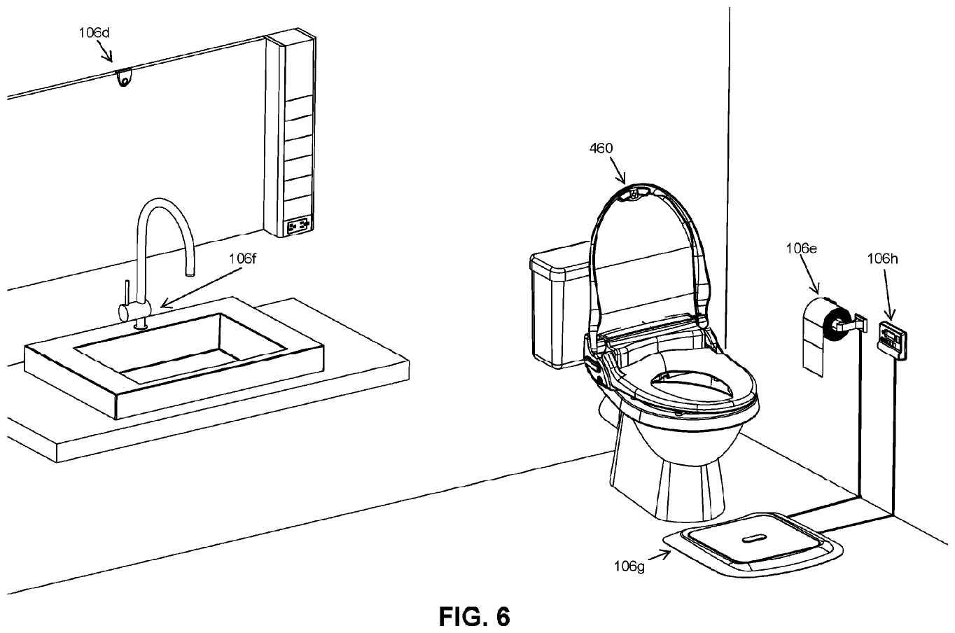

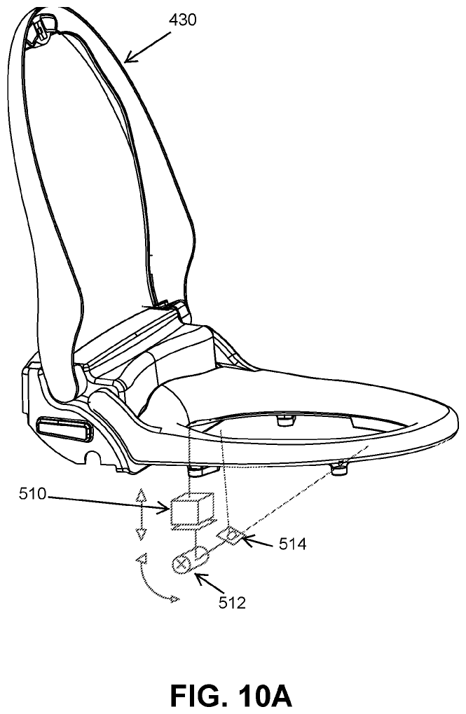

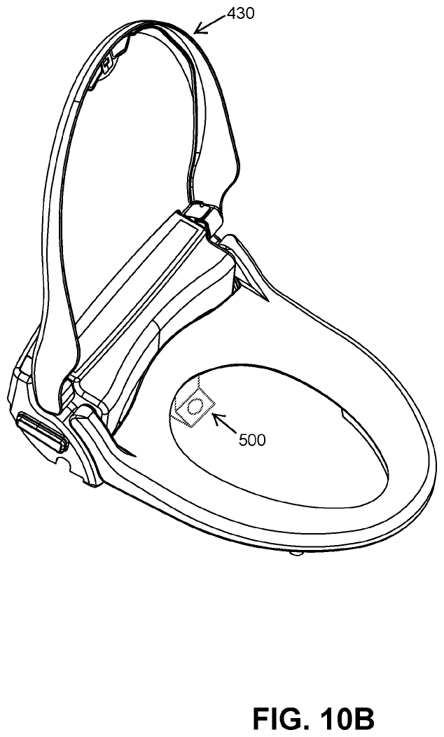

The sensors in the systems described herein can be located anywhere in the bathroom, e.g., near the BUAD. As illustrated in FIG. 6, examples of sensor locations include on a wall-mounted mirror 106d; a toilet paper roll 106e; a sink 106f; a mat in front of a toilet 106g; separately mounted on a wall 106h or installed or integrated into a seat or a seat cover on the toilet 460 (see also FIG. 10).

When housed in a toilet seat or id (cover), the sensors can take on a variety of electrode configurations for capacitive, bioelectrical impedance, and/or electrocardiogram measurements, as shown in FIG. 8. FIG.8A shows a single sensor on the top of the seat, represented by a rectangle. FIG. 8B shows four sensors on the top of the seat. FIGS. 8C, 8D, 8E, and 8F show various configurations of multiple sensors on the top of the seat. The electrodes could be incorporated into the seat or lid by any means, for example including chemical vapor deposition, sputtering, evaporation, inkjet printing, dip coating, screen printing, ultrasonic or laser welding of the module to the plastic, thus allowing electrical connections to be safely routed to control and sensing electronics. The electrodes may include specific biocompatible coatings to ensure good signal quality and no adverse user reaction.

FIGS. 9A, 9B. 9C, and 9D show embodiments where a sensor array 460 or a sensor 460b is situated on or in a lid/cover 430 such that parameters of the bathroom (e a visual image if at least one of the sensors is a camera) when the lid is lifted in preparation to use the toilet and an excretion analysis device 410 attached thereto. In FIG. 9A, a sensor array 460 is on the edge 432 of the lid 430 In these embodiments, the sensor array is comprised of recess 461, time-of-flight camera module 462, mount 463, lens cover 464 upon which coatings may be present that harden the material, provide anti-reflective properties that allow infrared light to pass through, are hydrophilic, are hydrophobic, and/or have anti-smudge properties and a rubber cover 465. At the hinge of the lid 440, there is a hinge cap 442 and cable 444 to allow for safe routing of electrical connections to control and sense electronics. An alternative embodiment is shown in FIG. 9B, where two sensors 460b , having either the same or different functionality, are near the top of a lid 430b . FIGS. 9C and 9D show an embodiment where the inner cavity 470 of the lid 430c houses electronics 480 to join the sensor(s) to the excreta analysis device 410 or a computational device.

In another implementation, the system can include an optical or thermal image sensor oriented upward in order to image the anus and genital region, to capture images which could be used to uniquely identify a user. FIGS. 10A and 10B illustrates examples of such a system that also comprises a sensor array on the lid as in FIG. 9A. In FIG. 10A, the upward facing system comprises an image sensor 510, a rotating mirror 512 and collection lens 514, such that the sensor therein can rotate to face upward when utilized. In an alternative embodiment, shown in FIG. 10B, the sensor 500 is stationary. In some embodiments, a series of mirrors and lenses are used to image upward from under the toilet seat.

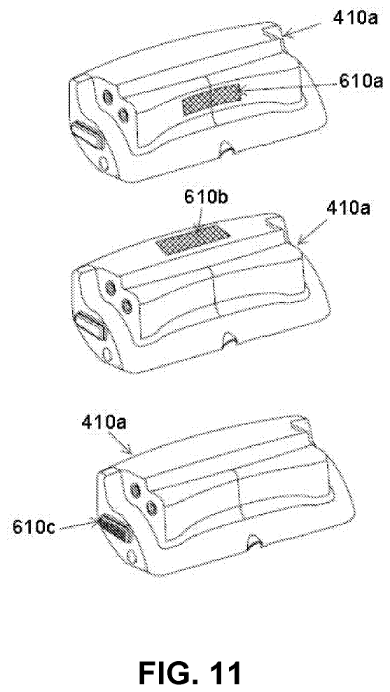

In further embodiments, the sensor(s) can be present on the BUAD. As an example, FIG. 11 shows a toilet with an excreta analysis device 410a where a sensor, for example, a fingerprint reader, is shown at three different positions 610a , 610b , 610c on the excreta analysis device 410a . Such systems can also include additional sensors, such as the sensor array 460 further described above and illustrated in FIG. 10A.

### User Profile Initialization

In various embodiments, the system is configured such that the user may be standing, sitting, or using an apparatus that makes it easier to use the appliance associated with the BUAD, such as toilet seat risers and support arms.

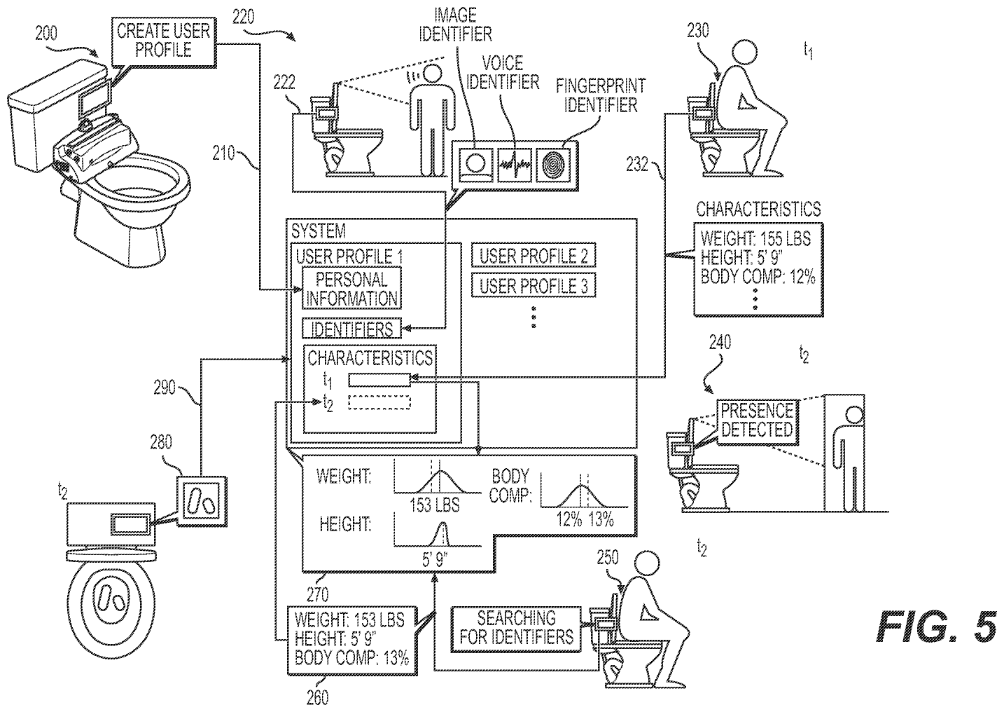

In embodiments of the system illustrated in FIG. 5, the system can generate a user profile 210 representing a user of the system. More specifically, the system can generate a user profile including personal information of the user in order to associate identifiers, characteristics, excretion events, and diagnostic information with a particular user. The system can generate the user profile via a native application executing on a smartphone, or other computational device of the user. Alternatively, the system can include a touchscreen or other input/output device to enable the user to input personal information for inclusion in the user profile. The system can provide a secure application programming interface (API) to add user profiles. The system can generate a user profile that includes the name, age, gender, medical history, address (e.g., for billing purposes), or any other information that is pertinent to analysis of the user's BUAD (in this example, an excreta analysis device) use. In order to collect personal information from the user, the system, at the BUAD or via a native application, can prompt the user to input any of the above-listed personal information and store the personal information in association with a UUID in a database located at the BUAD or on a server or another computing device connected to the BUAD.

In some embodiments, the system can associate the user profile with a specific BUAD in order to direct each particular BUAD to identify users of that particular BUAD.

### Acquiring User Identifiers

In the embodiment shown in FIG. 5, the system can prompt a new and/or first user to specify a first set of user identifiers 220; and associate the new and/or first user identifier with the new and/or first user profile of the user 222. More specifically, the system can prompt the new and/or first user to provide an identifier that the system can utilize to identify the new and/or first user with a high degree of confidence. In one implementation, the system can display—such as via an interface on the BUAD or via the native application executing on the user's mobile device a prompt to select from a predefined list of identifier options. Upon receiving a user selection corresponding to a particular identifier option, the system can provide an interface or execute a series of steps to record the identifier.

### User Characteristic Detection

As shown in FIG. 5, the system can measure a first set of user characteristics of the new and/or first user 230; and associate the first set of user characteristics with the new and/or first user profile 232. More specifically, the system can measure a set of user characteristics via the BUAD and/or other integrated sensors in order to characterize the user independent from the identifiers associated with the user (e.g., via sensor fusion), thereby improving the ability of the system to identify users. Therefore, in instances wherein the system cannot identify the user based on the set of identifiers associated with the user profile, the system can: measure characteristics of the current user; and match the set of characteristics of the current user with a set of characteristics associated with the user profile in order to identify the user.

In one implementation, during the onboarding process, the system can prompt the user to use the proximal toilet while recording the set of user characteristics corresponding to the user as the user uses the proximal toilet. Additionally or alternatively, the system can direct the user to position herself as if she were using the toilet in order to record a set of user characteristics of the user.

In one implementation, the system can save a set of user characteristics for each use of the BUAD and/or other integrated sensors. Over repeated measurements the system can distinguish between and among users based on patterns or similarities of the recorded user characteristics.

### Presence Detection

As shown in FIG. 5, after completing a new user profile for a new user during a first time period, the system can, during a later (second) time period detect the presence of a current user of the system 240. In specific embodiments, the system includes any or all of a time of flight camera, a passive infrared sensor (hereinafter a “PIR sensor”), a visual light camera, capacitance sensor, door switch, or any other sensor capable of detecting a presence of a current user. In response to detecting a presence of the current user, the system can prompt the user to provide an identifier from her user profile via an indicator light, touchscreen display, or audible message. In one implementation, the system activates a visual indicator that the user's presence has been detected indicating that the system is ready to record a BUAD use. In some embodiments, the system can detect the presence of a user standing in front of an excreta analysis device in preparation to urinate or the presence of a user sitting on the toilet seat of the excreta analysis device.

### User Identification

As shown in FIG. 5, the system can perform any or all of the following: in response to detecting presence of a current user, attempt detection of the first user identifier 250; in response to failure to detect the first user identifier, measure a set of current user characteristics 260; and matching the set of current user characteristics with the first set of user characteristics 270. More specifically, the system can execute identification logic in order to positively identify the current user of the BUAD or identify the current user as a guest user of the BUAD.

In response to detecting the presence of the user, in some embodiments the system can activate a camera (infrared or visual light) to record images of the detected user's face or body, a digital microphone to record the voice of the detected user, and/or a BLUETOOTH or WIFI chip to detect proximity of a known user device to the excreta analysis device. The system can also wait for an explicit identifier input from the user at a button or touchscreen of the excreta analysis device. In one implementation, the system continues detecting an identifier for the entire period during which the current user is detected proximal the BUAD.

In some embodiments, if the system detects an identifier such as facial image, body image, voice recording, direct input, fingerprint, and/or wireless ID of a user device, the system can match the detected identifier with the set of identifiers associated with the user profile in order to identify the current user. Additionally, as the user begins to use the BUAD, the system can simultaneously begin to measure a set of current characteristics of the user in order to identify the user if an identifier is not detected and to add to a corpus of characteristics for the current user upon identification of the current user. Furthermore, the system can record, in the form of images of the contents of the toilet, an excretion event as the current user uses the proximal toilet, while the system continues to gather a set of characteristics of the current user and attempts to detect identifiers of the current user.

### Method

As shown in FIG. 5, a method 200 for associating a BUAD use with a user includes any or all of the following steps: during a first time period, generating a new and/or first user profile representing a new and/or first user 210; prompting the new and/or first user to specify a first set of user identifiers 220; associating the new and/or first user identifier to the new and/or first user profile 222; measuring a first set of user characteristics of the new and/or first user 230; and associating the first set of user characteristics with the first user profile 232. During a second time period succeeding the first time period, in response to detecting presence of a current user 240, attempting detection of the first user identifier 250; and measuring a set of current user characteristics 260. The method 200 further includes, during the second time period and in response to matching the set of current user characteristics with the first set of user characteristics 270: at a BUAD, recording a BUAD use, e.g., an excretion event in a proximal toilet of an excreta analysis device 280; and associating the BUAD use with a user profile in 290.

As indicated above, in some embodiments, the bathroom use analysis device is an excreta analysis device that analyzes excreta during use of a toilet by the user. Any excreta analysis device, now known, or later discovered, can be incorporated into the systems provided herein. See also the various excreta analysis device embodiments in WO 2018/187790 (called biomonitoring devices therein). In various embodiments, the excreta analysis device analyzes urine, feces, flatus, or off-gas from feces or urine. In additional embodiments, the excreta analysis device comprises an excreta analysis sensor that detects electromagnetic radiation or an analyte chemical in a bowl of the toilet.

In some of these embodiments, the excreta analysis device comprises a urine receptacle, e.g., as described in U.S. Provisional patent application 62/959139 (“US 62/959139”). As exemplified therein, the urine receptacle can be disposable or reusable. In some embodiments, the excreta analysis device further comprises a replaceable visual urinalysis assay, e.g., a dipstick, as described in US 62/959139.

In additional embodiments, the excreta analysis device comprises a flushable stool collector, e.g., as exemplified at p. 9 and FIGS. 6A-C of WO 2018/187790.

In embodiments that identify specific users, the system utilizes a computational device that is capable of analyzing the data to determine characteristics of the user that are detected by the sensor. Various computer systems and data transmission formats are discussed in WO 2018/187790.

In some embodiments, the computational device is dedicated to user detection and identification and is joined to the sensor in a housing. In other embodiments, the computational device is not dedicated to user detection and identification and is not housed with the sensor.

In additional embodiments, data from the sensor is transmitted to the computational device by wire or by a wireless communication protocol.

In various embodiments, the computational device is also capable of analyzing data from the bathroom use analysis device, e.g., an excreta analysis device.

In accordance with various versions of the systems described above, the computational device comprises software that can use data from the sensor to detect and identify a first user, as well as detect and identify a different user. By repeating the protocols described above in a loop-like fashion, any number of users can be identified as users of the BUAD.

In an alternative implementation, the system can include an excreta analysis device that includes the toilet hardware, such as the bowl, tank, and other plumbing hardware.

In another implementation shown in FIG. 9A, the system includes a sensor cluster mounted on the top of the lid of a toilet and electrically coupled to the excreta analysis device such that the sensor cluster can capture images of users of the excreta analysis device.

In one implementation, the system can also include a user-interface—such as a touch screen display, a microphone, a speaker, indicator lights, a set of buttons, installed on the excreta analysis device, the proximal toilet, a toilet paper holder, a towel bar, and/or a support rail proximal the excreta analysis device in order to communicate with the user and receive inputs from the user.

In one implementation, a connected toilet paper roll holder is used to house user activation and identification sensors. The toilet paper roll holder can be configured to house a number of sensors including but not limited to an image sensor (visible and/or infrared), time of flight camera, LEDs or other light source, fingerprint reader, LCD touchscreen, and/or temperature sensors. In one implementation an Inertial Measurement Unit (IMU) is enclosed inside the arm holding the roll to measure the rotation and use of toilet paper. The recording of toilet paper use can be used for automatic toilet paper reordering or to distinguish users based on toilet paper consumption.

Also provided herewith is a method of detecting a user of a bathroom. The method comprises analyzing data generated by the sensor in any of the systems described above to detect and/or identify the user.

In some embodiment of these methods, data from the sensor is transmitted to a computational device that analyzes the data to detect and identify the user, as described above. In some of those embodiments, the computational device identifies the user by comparing the data from the sensor to data in a stored user profile, wherein, (a) if the data from the sensor matches the user profile, the user is identified as the user in the user profile, or (b) if the data from the sensor does not match the user profile or any other stored user profile, the user is identified as a guest or a new user, wherein the data from the sensor is used to create a user profile for the new user.

In some of these methods, the BUAD is an excreta analysis device.

In other embodiments of these methods, the system generates a user profile identifying an individual user; detects a presence of a current user; matches the current user with a user profile; records a bathroom use event; and associates the bathroom use event with the matched user profile. In further embodiments, the computational device or a second computational device analyzes data from the excreta analysis device and associates the data from the excreta analysis device with the user profile of the user.

Where the BUAD is an excreta analysis device, the present invention is not limited to the detection of any particular parameter or condition of the user. In various embodiments, the data from the excreta analysis device determines whether the user has a condition that can be discerned from a clinical urine or stool test, diarrhea, constipation, changes in urinary frequency, changes in urinary frequency, changes in urinary volume, changes in bowel movement frequency, changes in bowel movement volume, changes in bowel movement hardness, changes in urine color, changes in urine clarity, changes in bowel movement color, changes in the physical properties of stool or urine, or any combination thereof. See, e.g., WO 2018/187790.

In specific embodiments, the method is executed by an excreta analysis device—integrated with or including a toilet—and/or a set of servers (or other computational devices) connected to the excreta analysis device—in order to perform any or all of the following tasks: generate a user profile identifying an individual user; detect a presence of a current user proximal the excreta analysis device; match the current user of the system with the user profile; record an excretion event; and associate the excretion event with the matched user profile. Therefore, the system can associate a series of excretion events with an individual user over a period of time despite multiple users urinating and/or defecating in the toilet with which the system is integrated over the same period of time. As a result, the system, and/or a related system with access to the user-labeled series of excretion events, can analyze excretion events over time in order to statistically, including through machine learning, detect patterns in the user's excreta, thereby improving diagnosis of medical conditions or identification of gastrointestinal changes of the user.

In one implementation of the system data from sensors used for identification could be used to aid in the diagnosis of medical conditions, e.g., an electrocardiogram used to diagnose atrial fibrillation in a user. Another implementation of the system data from sensors used for identification could be used to aid in the measurement of gastrointestinal changes in the user, e.g., changes in heart rate during defecation. Another implementation of the system data from sensors used for identification could be used to aid in identifying a febrile user. Another implementation of the system data could be used to aid in monitor users for signs of infections or fevers.

The system can execute various parts of the method locally, e.g., at the BUAD, or remotely, e.g., at a computing device operatively connected to the BUAD. By selectively executing certain steps of the method either locally or remotely, and by executing encryption and other security features, the system can reduce the probability of linking potentially sensitive diagnostic information with the identity of the user by a malicious entity, while still enabling analysis of a series of BUAD uses associated with a particular user. Additionally, the system can interface with a user device via BLUETOOTH, Wi-Fi, NFC, or any other wireless communication protocol while executing parts of the method.

In various embodiments, the system can onboard new users of the BUAD by prompting the user to input identifying information such as the user's name, age, gender, etc. in order to generate a user profile for the user. Additionally, some embodiments of the method can prompt the user to specify a first set of identifiers, such as explicit identifiers (e.g., button presses or touchscreen interaction at the excreta analysis device), voice identifiers (e.g., sample audio clips for identification of the user), image identifiers (e.g., a set of images of the users face or body), structured-light 3D scanning identifiers (e.g., measuring the three-dimensional shape of a face or body using projected light patterns and a camera system), fingerprint identifiers, retinal identifiers, smartphone/wearable identifiers (e.g., a BLUETOOTH ID of the user's smartphone or wearable device) as previously discussed. Therefore, the system, upon detecting an identifier or a combination of identifiers in the set of specified identifiers corresponding to a particular user, can positively identify the particular user of the BUAD at the time of detection.

During the onboarding process or a subsequent BUAD use positively identified as corresponding to an existing user profile, some embodiments of the method can also measure and record a set of physical characteristics of the user such that the system can identify the user in the absence of any of the specified identifiers of the user. As previously discussed, the method can record physical characteristics, such as the user's height, weight, weight distribution on the proximal toilet of the excreta analysis device, skin color, heart rate, electrocardiogram, temperature, bioelectrical impedance, and associate these characteristics with the user profile. These embodiments of the method can, therefore, match characteristics of future users of the excreta analysis device to the set of characteristics associated with a user profile in order to identify the user when, for example, the user forgets their phone or is unable to communicate due to cognitive decline (e.g., dementia), does not present their face to a camera of the excreta analysis device, or does not respond to a voice prompt to identify herself, thereby preventing direct identification of the user.

While the method attempts to identify the current user of the BUAD, some embodiments of the method can record an excretion event of the current user at the BUAD and store any recorded optical data or other data representing the BUAD use. Upon identification of the current user, the method can associate the BUAD use with the user profile corresponding to the identity of the current user. However, in some implementations, the method can store a BUAD use with no associated user profile in association with any measured characteristics of the user responsible for the excretion event. Therefore, upon recording a threshold number of BUAD uses associated with a sufficiently similar set of characteristics (e.g., within a threshold similarity), the method can create an unidentified user profile and prompt the anonymous user responsible for the excretion events to enter a user information at the excreta analysis device.

The system and the method are hereinafter described with reference to a “first user.” However, the system can also support additional users (second, third, etc.) by repeatedly executing parts of the method in order to generate multiple user profiles thereby supporting multiple concurrent users of the excreta analysis device.

Upon completion of the BUAD use or in response to the system detecting an absence of the current user near the BUAD, the system can evaluate any detected identifiers and/or detected characteristics according to the identification logic shown in FIG. 7.

In the FIG. 7 exemplary implementation, the system first detects the presence of the current user 300. The system evaluates whether it has detected any identifiers that match the set of identifiers associated with the user profile of a first user 310 and determines whether an identifier is detected 320. For example, if the system records an image of the face of the current user, then the system can perform facial recognition techniques to match the face of the current user to image identifiers stored in association with the user profile. In another example, if the system records an audio clip of the current user, the system can match the audio recording to the voice identifiers stored in association with the user profile according to voice recognition techniques. In another example, if the system records a direct interaction with a button or touchscreen of the BUAD, the system can identify the corresponding user profile that is assigned to the button or touchscreen input. In yet another example, if the system records a fingerprint at a fingerprint scanner of the excreta analysis device, the system can match the recorded fingerprint to a fingerprint identifier stored in association with the user profile.

If the system fails to identify the user via an identifier 330, as described above, the system can match a set of recorded characteristics of the current user to the set of characteristics stored in association with the user profile 350. In one implementation, the system can calculate a probability distribution based on typical or observed variation of each characteristic of a first user and, upon measuring a characteristic of a current user, calculate the probability of the current user matching the first user based on the probability distribution. The system can repeat this process for each characteristic in the set of characteristics and calculate a total probability of a match between the first user and the current user. In response to calculating a total probability of a match greater than a threshold probability, the system can identify the current user as the first user.

In this implementation, the system can define probability distributions for specific users and/or for specific individuals. For example, the system can define a narrow distribution for a user's height, since height is not expected to vary outside of measurement error, while defining a wider distribution for a user's weight since the expected variation in a user's weight is often about 1% of her average weight. In another example, the system can store a time series of each characteristic of the user and calculate a probability distribution based on the time series of each characteristic. For example, the system can calculate a standard deviation of the user's weight, as measured by the excreta analysis device over several excretion events and calculate a probability distribution for the user's weight during a subsequent excretion event. Additionally, the system can calculate a probability distribution weighted by the recency of previously measured characteristics by, for example, calculating a weighted standard deviation or a weighted average of previously measured characteristics; and calculating a probability distribution for the characteristics based on the weighted standard deviation or the weighted average. Furthermore, the system can increase the width of the probability distribution for a particular characteristic based on the amount of time since the last excretion event attributed to the user, since variation in characteristics such as the user's weight may be expected to increase over longer periods of time.

In another implementation, the system can utilize a machine/deep learning model in order to identify the user by classifying the user from amongst a set of known user profiles. For example, the system can execute an artificial neural network defining two input vectors to the network: one for a user profile and another for characteristics recorded for a current user. The system can then execute the network to calculate a confidence score that the characteristics of the current user match the user profile. In one implementation, the system trains the machine/deep learning model based on previous instances of the system recording characteristics of the user.

In additional embodiments, the system can match a current set of user characteristics to a stored set of user characteristics by executing any statistical or machine/deep learning classification algorithm.

As shown in FIG. 7, if the system fails to match an identifier of a current user to an identifier associated with a user profile 330 and fails to match the set of characteristics of the current user to a set of characteristics associated with a user profile 340, the system can classify the user as a guest user and store the excretion event data in association with the guest user 340.

### Excreta Analysis

As shown in FIG. 5, some embodiments of the system can: at the excreta analysis device, record an excretion event in the proximal toilet of the excreta analysis device 280; and associate the excretion event with the first user profile 290. More specifically, in various embodiments, the system can capture images and spectral data collected via selective laser and/or LED excitation of the user's excreta. In further embodiments, the system can label images and other data recorded at the excreta analysis device based on the presence of feces, urine, and toilet paper. Upon identification of the user responsible for the excretion event, the system can store the associated images and data of the excretion event in association with the user profile. The system can then analyze these data over multiple excretion events in order to improve the user's health/wellness or diagnose gastrointestinal conditions of the user via image analysis, machine learning, and other statistical tools.

Therefore, in one implementation, the system can: store an unidentified excretion event with a corresponding set of user characteristics; generate a guest user profile based on the set of user characteristics; and associate the unidentified excretion event with the guest user profile. Therefore, the system can identify new users of the excreta analysis device and track excretion events before or without explicit onboarding the user. Thus, when the anonymous user does create a profile with the system, the system has already recorded excretion event data and characteristics of the user and can immediately deliver any diagnostic results or insights to the new user.

Additionally, the system can attempt to match subsequent unidentified users with the previously generated guest profile(s). If the system calculates a high probability of a match between measured characteristics of an unidentified user and a set of characteristics associated with a guest user profile, the system can store the excretion event corresponding to the unidentified user with the guest user profile.

In one implementation, the system can, in response to recording a threshold number of excretion events associated with a guest user profile, prompt the guest user (upon detecting the presence of the guest user immediately prior to, during, and/or after an excretion event) to create a user profile with the system. In response to the guest user responding to this prompt (via an input at an interface of the excreta analysis device or at a native application), the system can begin the above-described onboarding process.

In another implementation, the system can, in response to failure to identify a current user, prompt a known user of the excreta analysis device (e.g., via a native application on the user's personal device) to verify whether she is responsible for a recent excretion event. For example, if the system is unable to identify a current user during an excretion event, the system can send a notification to a user's smartphone requesting the user to verify whether she just used the proximal toilet. In response to receiving an input from the user affirming that she did use the proximal toilet, the system can associate the excretion event with the known user. In response to receiving an input from the user denying that she used the proximal toilet, the system can generate a guest user profile for the set of characteristics of the current user corresponding to the excretion event.

In yet another implementation, the system can discard excretion event data upon failure to identify the current user in order to mitigate privacy concerns.

### Privacy Features

Because the system handles potentially embarrassing and private information, some embodiments of the system can execute privacy features to obscure diagnostic information, identifying information, BUAD use related information (such as raw images of excreta or the timing of a user's bowl movements). Thus, the system can execute specific parts of the method locally, at the BUAD, or remotely, at a server connected to the BUAD in order to reduce the likelihood of sensitive data from being intercepted in transit or present at a decentralized location such as the BUAD. Additionally, some embodiments of the system can schedule and/or batch transmissions between the excreta analysis device and the set of servers in the system while transmitting identifying information and diagnostic information separately, thereby obscuring the timing of particular excretion events and the associated identify of a user responsible for the particular excretion event. Furthermore, various embodiments of the system can encrypt all transmissions between the excreta analysis device and remote servers of the system.

In one implementation, the system executes analysis of BUAD use at the BUAD and sends resulting diagnostic information to a remote server. The system can then also send identifiers and characteristics of the user recorded in association with the diagnostic information. The remote server can then identify the user associated with the diagnostic information. Therefore, in those embodiments, the system does not send images of excreta, thereby preventing interception of these images by a malicious actor. Alternatively, the system can prioritize the security of diagnostic information and perform diagnostic analysis of excreta images at a remote server, thereby preventing transmission of diagnostic information between the excreta analysis device and the set of remote servers.

In another implementation, the system batches identifying information (identifiers and characteristics of users) and excreta images and/or diagnostic information and transmits this information to remote servers for further analysis on a predetermined schedule. Additionally or alternatively, the system can transmit identifying information separately from diagnostic information and/or excreta images in order to prevent association of diagnostic information and/or excreta images with the identity of a user by a malicious actor. For example, the system can transmit data between the excreta analysis device and the set of remote servers at two different times, once to transmit identifying information for particular excretion events, and a second time to transmit diagnostic information and/or excreta images. The system can then relate these disparately transmitted data at the remote server according to identification labels not associated with a user profile.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

### REFERENCES

PCT Patent Publication WO 2018/187790

U.S. Provisional Patent Application No. 62/809522.

U.S. Provisional Patent Application No. 62/900309.

U.S. Provisional Patent Application No. 62/959139.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A system for detecting a user of a bathroom, the system comprising at least one sensor coupled to a bathroom use analysis device, wherein the sensor generates data that can be used to detect and/or identify the user.

2. The system of claim 1, wherein the sensor comprises an explicit identifier, an image sensor, a time of flight camera, a load cell, a capacitive sensor, a microphone, an acoustic sensor, a sonic sensor, an ultrasonic sensor, a passive infrared sensor, a thermopile, a temperature sensor, a motion sensor, an ambient light sensor, a photoelectric sensor, a structured light system, a fingerprint scanner, a retinal scanner, an iris analyzer, a smartphone, a wearable identifier, a scale integrated with a bathroom mat, a height sensor, a skin color sensor, a bioelectrical impedance circuit, an electrocardiogram, a pulse oximeter, a thermometer, or any combination thereof.

3. The system of claim 1, comprising more than one sensor that generates data that can be used to detect and/or identify the user.

4. The system of claim 1, wherein the bathroom use analysis device analyzes activity at a mirror, sink, a tub, a shower, a medicine cabinet, a toilet, a bidet, or any combination thereof.

5. The system of claim 1, wherein the bathroom use analysis device is an excreta analysis device that analyzes excreta during use of a toilet by the user.

6. The system of claim 5, wherein the excreta analysis device analyzes urine, feces, flatus, or off-gas from feces or urine.

7. The system of claim 5, wherein the excreta analysis device analyzes urine.

8. The system of claim 5, wherein the excreta analysis device analyzes feces.

9. The system of claim 5, wherein the excreta analysis device analyzes urine and feces.

10. The system of claim 5, wherein the excreta analysis device comprises an excreta analysis sensor that detects electromagnetic radiation or an analyte chemical in a bowl of the toilet.

11. The system of claim 5, wherein the excreta analysis device comprises a urine receptacle.

12. The system of claim 11, wherein the urine receptacle is disposable.

13. The system of claim 11, wherein the urine receptacle is reusable.

14. The system of claim 11, wherein the excreta analysis device further comprises a replaceable visual urinalysis assay.

15. The system of claim 14, wherein the replaceable visual urinalysis assay comprises a dipstick.

16. The system of claim 5, wherein the excreta analysis device comprises a flushable stool collector.

17. The system of claim 1, wherein the sensor comprises an image sensor, a time of flight camera, a load cell, a temperature sensor, ultrasound sensor, capacitance sensor, or any combination thereof.

18. The system of claim 5, wherein the sensor is an image sensor.

19. The system of claim 18, wherein the image sensor is a time-of-flight camera.

20. The system of claim 18, wherein the image sensor is installed above a seat or a seat cover on the toilet.

21. The system of claim 18, wherein the image sensor is installed or integrated into a seat or a seat cover on the toilet.

22. The system of claim 21, wherein the image sensor is integrated or installed onto the seat cover on the toilet, wherein the image sensor is capable of imaging the user only when the seat cover is raised.

23. The system of claim 21, wherein the image sensor is integrated or installed between the seat cover and the seat such that the image sensor is capable of imaging the user only when the seat cover is raised.

24. The system of claim 5, wherein the sensor is more than one load cell integrated into feet on a bottom of a seat on the toilet, wherein the load cells measure weight distribution of the user on the toilet.

25. The system of claim 1, wherein data from the sensor is transmitted to a computational device, wherein the computational device is capable of analyzing the data to determine characteristics of the user that are detected by the sensor.

26. The system of claim 25, wherein the computational device is dedicated to user detection and identification and is joined to the sensor in a housing.

27. The system of claim 25, wherein the computational device is not dedicated to user detection and identification and is not housed with the sensor.

28. The system of claim 27, wherein data from the sensor is transmitted to the computational device by wire or by a wireless communication protocol.

29. The system of claim 27, wherein the computational device is also capable of analyzing data from the bathroom use analysis device.

30. The system of claim 29, wherein the bathroom use analysis device is an excreta analysis device.

31. The system of claim 25, wherein the computational device comprises software that can use data from the sensor to detect and identify a first user, as well as detect and identify one or more different additional users.

32. The system of claim 31, wherein the software can generate a first user profile for the first user, and a second user profile for the second user.

33. A method of detecting a user of a bathroom, the method comprising analyzing data generated by the sensor in the system of any one of claims 1-32 to detect and/or identify the user.

34. The method of claim 33, wherein data from the sensor is transmitted to a computational device that analyzes the data to detect and identify the user.

35. The method of claim 34, wherein the computational device identifies the user by comparing the data from the sensor to data in a stored user profile, wherein, (a) if the data from the sensor matches the user profile, the user is identified as the user in the user profile, or (b) if the data from the sensor does not match the user profile or any other stored user profile, the user is identified as a guest or a new user, wherein the data from the sensor is used to create a user profile for the new user.

36. The method of claim 34, wherein the bathroom use analysis device is an excreta analysis device.

37. The method of claim 34, wherein the system generates a user profile identifying an individual user; detects a presence of a current user; matches the current user with a user profile;
records a bathroom use event; and associates the bathroom use event with the matched user profile.

38. The method of claim 37, wherein the bathroom use analysis device is an excreta analysis device.

39. The method of claim 38, wherein the computational device or a second computational device analyzes data from the excreta analysis device and associates the data from the excreta analysis device with the user profile of the user.

40. The method of claim 39, wherein the data from the excreta analysis device determines whether the user has a condition that can be discerned from a clinical urine or stool test, diarrhea, constipation, changes in urinary frequency, changes in urinary frequency, changes in urinary volume, changes in urine color, changes in bowel movement frequency, changes in bowel movement volume, changes in bowel movement hardness, changes in bowel movement color, or any combination thereof.
